(19) United States
(12) Patent Application Publication
(10) Pub. No.: US 2006/0102737 A1
(43) Pub. Date: May 18, 2006

(54) Volatile liquid dispensing device and method for releasing volatile compounds using a volatile liquid dispensing device

(75) Inventors: William M. Harmon, DeForest, WI (US); Kurt A. Windus, Madison, WI (US)

Correspondence Address: LATHROP & CLARK LLP, 740 REGENT STREET SUITE 400, P.O. BOX 1507, MADISON, WI, 537011507, US

(73) Assignee: G.B.O.W. Hunting Solutions LLC, Portage, WI (US)

(21) Appl. No.: 10/972,701

(22) Filed: Oct. 25, 2004

(51) Int. Cl. A61L 9/04

(52) U.S. Cl. 239/6; 239/53; 239/34

(57) ABSTRACT

A device for dispensing volatile liquids has a head and a body and can be formed of an absorbent material. A volatile liquid is applied to the body of the device so that the body absorbs the volatile liquid. To attach to or engage the volatile liquid dispensing device with a support structure, a tab, cut into the head of the device, is pulled out from the head along a spiral incision and engaged with a support structure. The recoil inherent in the spiral incision further secures the device to the support structure. As the liquid evaporates, the volatile liquid disperses into the atmosphere.

### BACKGROUND OF THE INVENTION

1. Field of the Invention

This invention relates to devices and methods used to release volatile compounds into an atmosphere.

2. Related Art

Releasing volatile compounds, such as aromas and scents, into a selected environment is becoming a widely used method of modifying that environment for a variety of purposes. Such scents may be used both indoors and outdoors. For example, aromatherapy is widely used to release volatile compounds into a person's surroundings to take advantage of a scent's desirable or healing qualities. Other scents, used outdoors, are released into the environment to repel pests, attract desirable animals or to mask the presence of undesirable or offensive odors.

Hunters' use of scent devices outdoors is illustrative of the multiple uses of scents. A hunter may use a scent device to release attractant scents as a lure for desired game, to release repellant scents to keep away pests or undesirable animals or to release masking scents that hide undesirable odors, such as the hunters own scent. Examples of scents used include: the urine of a deer in-rut to attract bucks, citronella to repel mosquitoes and the use of sage to mask the hunters own odor.

Scent dispensing devices come in many forms. For example, some scent dispensing devices, such as that disclosed in U.S. Pat. No. 6,085,026, are designed to be plugged into an electrical outlet to volatize the scent. Other portable scent dispensing devices, such as car air-freshener devices, comprise a scent-impregnated matrix having some desirable shape and are hung by a string from a car's rearview mirror. Some scent dispensing devices operate mechanically, such as those described in U.S. Pat. No. 6,470,619, which resembles the reel-portion of a fishing reel, and in which a scented cord is reeled in after the cord is dragged along a forest path to attract game. U.S. Pat. No. 6,158,668 and Published U.S. Patent Application 2004/0050950 both describe scent dispensing devices that comprise closed containers having an outer housing, including an end-cap that can be opened to allow an interior wick to be exposed to the atmosphere.

In use, these portable scent dispensing devices have a cord or other closed-loop-type hanging structure that is hung over a support structure allowing the volatile liquid to be presented to the atmosphere for dispersal. Other scent dispensing devices, such as that disclosed in U.S. Pat. No. D435,896 to Burgeson, comprise an absorbent wick having a body on one end and a head with a hole on the other end. The hole allows an appropriately sized twig or branch to be inserted through the hole. The scent dispensing device hangs from the twig or branch, allowing the scent to be dispersed into the environment.

### SUMMARY OF DISCLOSED EMBODIMENTS

While these known scent dispensing devices aid in dispensing volatile scents to the environment, they are all limited by their ability to be deployed on support structures when dispersing the volatiles into the atmosphere. In environments which do not provide an easily usable support structure for the scent dispensing device, the scent dispensing device cannot be advantageously presented to the air to disperse volatiles into the atmosphere.

This invention provides a scent dispensing device having a head and body, the head having a spiral incision.

This invention separately provides a scent dispensing device having an engaging structure that allows the scent dispensing device to be deployed on various support structures.

This invention separately provides a scent dispensing device having an inner tab formed by a spiral incision cut in a head portion that allows the scent dispensing device to engage a support structure.

This invention separately provides a scent dispensing device having a thickness sufficient to provide an engaging structure with an elastic recoil which aids in securing the scent dispenser to the support structure.

This invention separately provides a scent dispensing device to which scent is easily applied.

This invention separately provides a scent dispensing device that easily adsorbs and/or absorbs scent.

This invention separately provides a scent dispensing device that is camouflaged to the environment.

In various exemplary embodiments, the scent dispensing device according to this invention has a body to which scent is easily applied. In various exemplary embodiments, the scent dispensing device has a head with an engaging structure, the engaging structure including an inner tab formed by a spiral incision cut into the head of the scent dispensing device. In various exemplary embodiments, the scent dispensing device according to this invention has a thickness that allows the inner tab to act as a projecting member when the scent dispensing device is engaged on a variety of support structures. In use, the body is dipped in a container of scent, the inner tab is pulled out from the head resulting in an engaging structure and the engaging structure is then easily engaged to a support structure allowing the scent to be dispersed into the selected environment

These and other features and advantages of various exemplary embodiments of the scent dispensing device and methods according to this invention are described in, or are apparent from, the following detailed description of various exemplary embodiments of the article and methods according to this invention.

### BRIEF DESCRIPTION OF THE DRAWINGS

Various exemplary embodiments of the structures according to this invention will be described in detail, with reference to the following figures, wherein:

FIG. 1 is a front elevation view of one exemplary embodiment of the scent dispensing device according to this invention;

FIG. 2 is a front perspective view of the scent dispensing device of FIG. 1;

FIG. 3 is perspective view of the scent dispensing device of FIG. 1 engaged to a horizontal support structure;

FIG. 4 is a perspective view of the scent dispensing device of FIG. 1 engaged to vertical support structure; and

FIG. 5 is a flow chart outlining one exemplary embodiment of a method for dispensing a volatile liquid into an environment according to this invention.

### DETAILED DESCRIPTION OF EXEMPLARY EMBODIMENTS

The following detailed description of some exemplary embodiments of a volatile liquid dispensing device according to this invention will generally focus on a particular use—to dispense scent into an ambient environment. As such, the volatile liquid dispensing device according to this invention will be specifically referred to as a scent dispensing device. Nevertheless, it should be understood that any volatile liquid, beyond merely scents, that a user wishers to dispense into an ambient environment can be dispensed using one or more embodiments of the volatile liquid dispensing device according to this invention.

While there is no shortage of different conventional scent dispensing devices, all are limited in their ability to be engaged to an appropriate support structure by the user. The following detailed description discloses various exemplary embodiments of a scent dispensing device that allows the scent dispensing device to be easily deployed in desired locales and engaged with a support structure so that the scent can be advantageously released into the atmosphere.

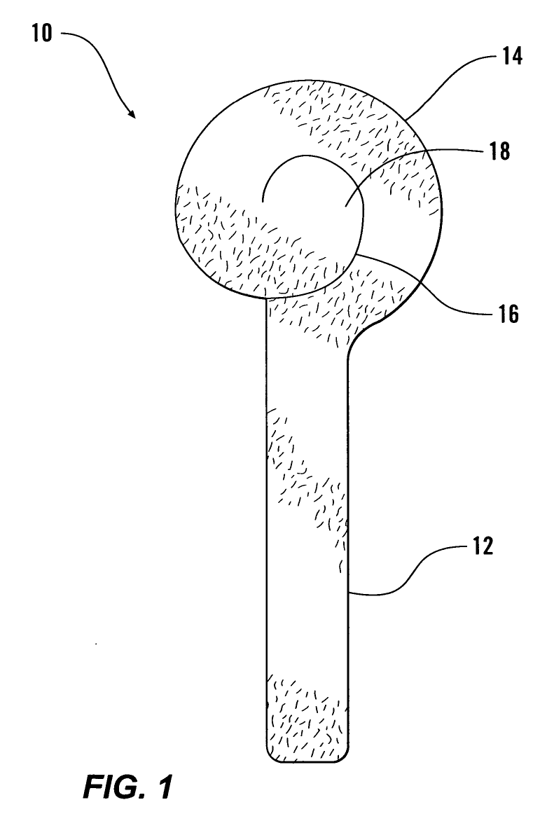

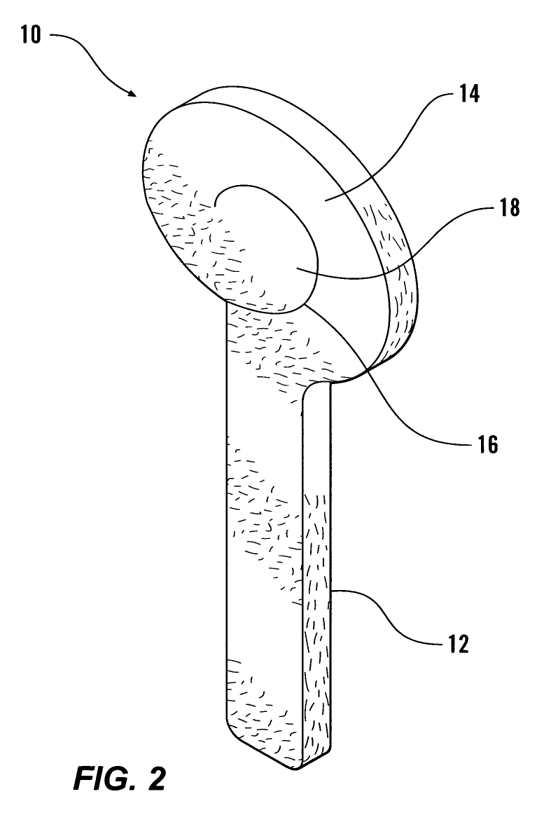

FIG. 1 is a front elevation and FIG. 2 is a perspective view of one exemplary embodiment of a scent dispensing device 10 according to this invention. As shown in FIGS. 1 and 2, in this exemplary embodiment, the scent dispensing device 10 is roughly key-shaped and has a body 12 connected to a head 14. In this exemplary embodiment, the head 14 of the scent dispensing device 10 is roughly circular in shape and resembles a fiddle-head having a spiral incision 16 cut in the head 14. In the exemplary embodiment shown in FIGS. 1 and 2, the spiral incision 16 is cut so that the spiral incision 16 creates an inner tab 18 in the head 14 of the scent dispensing device 10. In particular exemplary embodiments, the total length of the scent dispensing device 10 is about 4.25 inches, with the length of the body 12 about 2.75 inches and the diameter of the head 14 about 1.5 inches. However, it should be appreciated that the scent dispensing device 10 can be any size that is conveniently carried and engaged to a support structure.

The thickness of the scent device 10 is further illustrated in the perspective view shown in FIG. 2. In this exemplary embodiment, the recoil inherent in the spiral incision 18 is reinforced by providing a scent dispensing device 10 that has a thickness of approximately 0.25 inches. In addition, the scent dispensing device 10 has an inherent strength that makes the scent dispensing device 10 essentially self-supporting when engaged to a support structure. In addition, by providing a scent dispensing device 10 having a thickness of about 0.25 inches, the scent dispensing device 10 is able to absorb or adsorb a greater volume of liquid scent. Further, after the liquid scent is absorbed or adsorbed by the scent dispensing device 10 and the scent dispensing device 10 engaged with a support structure, the evaporation of the volatile liquid can cause the material of the scent dispensing device 10 to contract, enhancing the engagement of the scent dispensing device 10 with the support structure.

In use, the body 12 of the scent dispensing device 10 may be dipped in a container of volatile liquid scent that is to be dispersed or released into the environment. After the scent is applied to the scent dispensing device 10, the inner tab 18 is pulled out from the head 14 to unwind the head 14, so that the head 14 of the scent dispensing device 10 forms a hook-like engaging structure. The head 14 can then be wound or hooked around any appropriately-sized support structure, such as the branch of a tree or a vertical support structure, such as the trunk of a sapling. Because there is some recoil inherent in the spiral cut formed by the incision 16, the scent dispensing device 10 remains securely fastened to the support structure. In addition, for support structures having a relatively large diameter, the head 14 will deform as it is wrapped around the support structure, causing the inner tab 18 to act like a projecting member as it rests against the support structure.

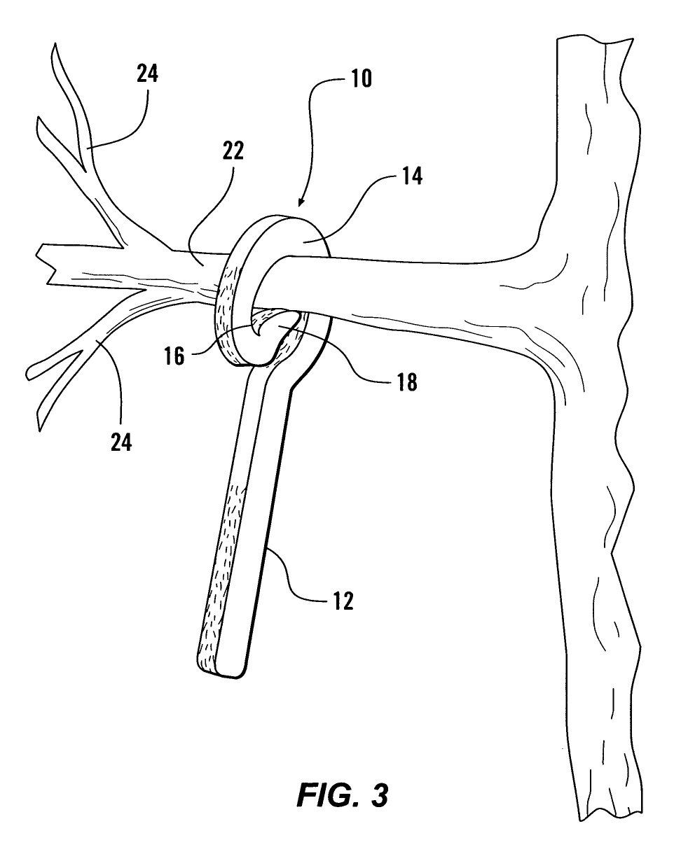

FIG. 3 illustrates the scent dispensing device 10 when deployed on a horizontally oriented support structure 22. As can be appreciated, due to the spiral incision 16 cut into the head 14, the scent dispensing device 10 is easily attached to a support structure 22. As shown, the support structure 22 has side branches 24 and does not have a readily available end or tip over which a closed-ended loop or hole could be placed. FIG. 3 also illustrates how the inner tab 18 is displaced against the support structure 22 such that the inner tab 18 results in an increase in the surface area that bears against the support structure 22. Increasing the surface area of the scent dispensing device 10 that bears on the support structure 22 helps to maintain the stability of the scent dispensing device 10 on the support structure 22.

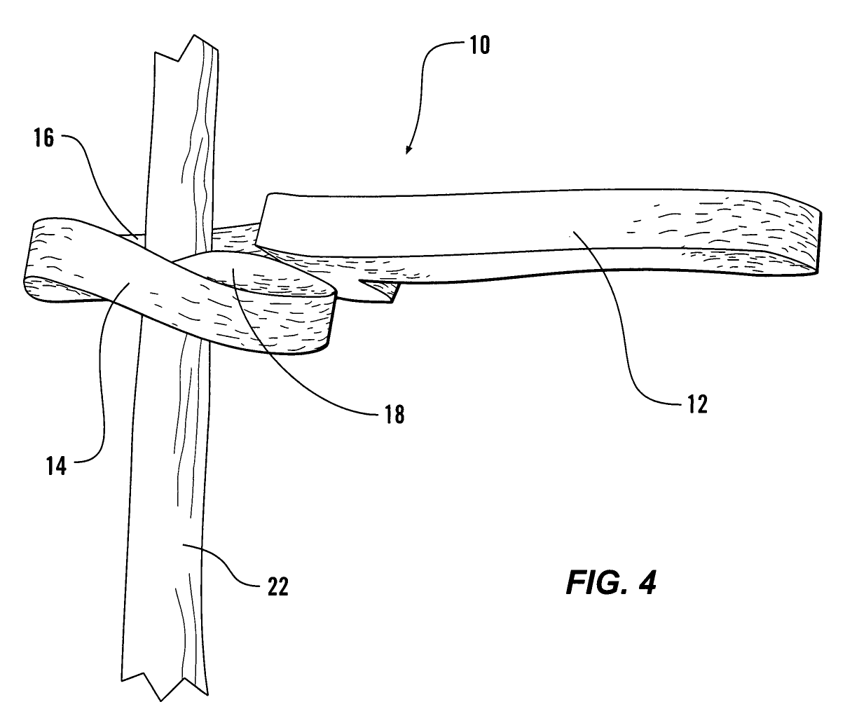

FIG. 4 shows the scent dispensing device 10 attached to a vertically oriented support structure 22. As shown in FIG. 4, the scent dispensing device 10 is also easily engaged to a vertical support structure, such as the trunk of a sapling. In this position, the thickness of the scent dispensing device 10 allows the scent dispensing device to be self-supporting insuring greater exposure to the atmosphere as illustrated in FIG. 4. In addition, various scent dispensing devices according to this invention can be secured to a bundle of pine needles, a bundle of leaves, a bundle of tall grasses or the like, depending on the available support structures in any given locale.

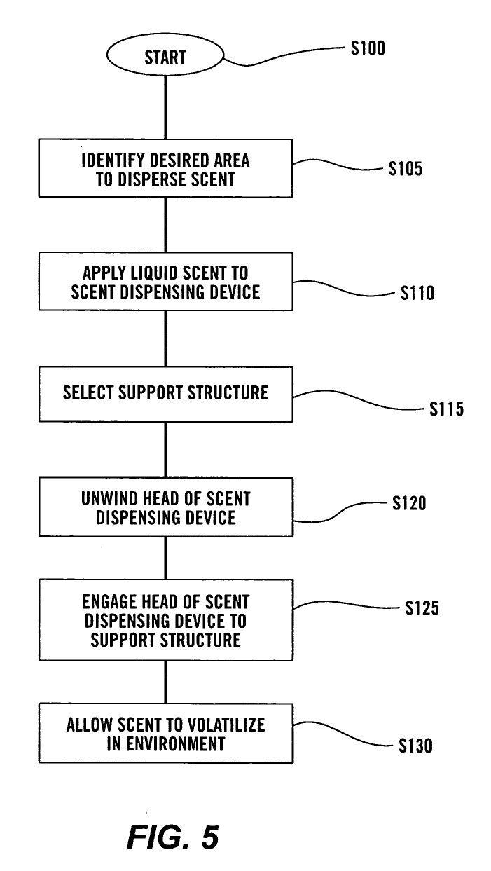

FIG. 5 is a flow chart outlining one exemplary embodiment of a method for using a scent dispensing device according to this invention. As shown in FIG. 5, the method begins in step S100 and continues to step S105, where the area desired by the user to disperse the scent in is identified. As outlined above, the scent dispensing device can be used both indoors and outdoors. However, when used in hunting to attract game, for example, the area identified in step S105 would be a hunting area. Next, in step S110, a volatile liquid, such as a scent, is applied to the scent dispensing device. When the scent dispensing device is used by a hunter to dispense scent, the scent may be the urine of a deer-in-rut; accordingly, the hunter may carry the urine in a closed container about his person. To apply the scent, the hunter would open the container of urine and dip the body of the scent dispensing device into the container. Then, in step S115, the user selects an appropriate support structure. When used by a hunter, the support structure may be the branch of a tree. It should be appreciated, in general, that the higher off the ground the support structure is, the more rapidly the scent will disperse into the atmosphere. Next, in step S120, the user unwinds the head of the scent dispensing device to free the tab. Then, in step S125, the scent dispensing device is engaged with the support structure by wrapping the head around the support structure. Finally, in step S130, the user allows the volatile scent to disperse into the environment.

In various exemplary embodiments, the scent dispensing device 10 is made of unbleached wool felt. In other exemplary embodiments, the scent dispensing device 10 can be made of other materials, such as paper-fiber, cardboard, cellulose fibers, cotton, artificial fibers such as nylon or Dacron, or the like. In general, any material that will absorb, adsorb or otherwise carry a suitable amount of a desired volatile liquid can be used. Similarly, it should be appreciated that while, in various exemplary embodiments, the incision 16 is a spiral the incision 16 can be any other desired shape, so long as the head 14 can be unwound or otherwise manipulated to engage the scent dispensing device with a support structure.

In addition, it should be appreciated that the scent dispensing device according to this invention can be used for the delivery of any appropriate volatile liquid, including scents, aromas or odors, into any desired environment. For example, the scent dispensing device can be used outdoors by a hunter to disperse an attractant, repellant or masking scent. Alternatively, the scent dispensing device can be used indoors or outdoors to dispense air fresheners, deodorizers or other desirable scents and/or other desirable volatile liquids into a desired ambient atmosphere. For example, the scent dispensing device according to this invention can be used to dispense any number of scents that are marketed as aromatherapy oils to facilitate pleasant or healing aromatherapy. Further, the scent dispensing device according to this invention, may also have a core or be impregnated with activated carbon or other scent or odor-absorbing material. In this exemplary embodiment, the scent dispensing device would be deployed in the selected environment as described so that the undesirable odors could be absorbed from the environment.

It should be appreciated that the scent dispensing device according to this invention can have any number of shapes so long as it provides a head having an inner tab that acts as a support member and a body that can absorb, adsorb or otherwise carry a desired quantity of the volatile liquid. For example, the head may be squared, polygonal, circular, elliptical, oval or the like. In some exemplary embodiments, by providing a decreasingly concentric incision around the periphery of the head, the head will form an engaging structure that can be easily engaged with a support structure. Similarly, while the exemplary embodiments outlined above describe the incision as extending to the edge of the head, allowing the tab to be pulled free from the head, it should be appreciated that the incision may not be cut to the edge of the head. In such exemplary embodiments, the tab would pull out from the middle of the head to create the engaging structure.

The above-outlined exemplary embodiments of the scent dispensing device according to this invention are usable to secure a scent dispensing device to a support structure either in the vertical or horizontal position. Each of the various structures and methods described herein are separately useful.

While this invention has been described in conjunction with the exemplary embodiments outlined above, various alternatives, modifications, variations, improvements, and/or substantial equivalents, whether known or that are or may be presently unforeseen, may become apparent to those having at least an ordinary skill in the art. Accordingly, the exemplary embodiments of the invention as set forth above are intended to be illustrative, not limiting. Various changes may be made without departing from the spirit and scope of the invention. Therefore, the invention is intended to embrace all known or earlier developed alternatives, modifications, variations, improvements and/or substantial equivalents.

What is claimed is:

1. A volatile liquid dispensing device comprising:
a body; and
a head, wherein the head has an incision cut into the head.

2. The volatile liquid dispenser of claim 1, wherein the incision forms an inner tab portion of the head.

3. The volatile liquid dispensing device of claim 2, wherein the incision is in the shape of a spiral.

4. The volatile liquid dispensing device of claim 2, wherein the incision extends to the edge of the head.

5. The volatile liquid dispensing device of claim 1, wherein, in use. a volatile liquid is applied at least to the body.

6. The volatile liquid dispensing device of claim 5, wherein the volatile liquid is an animal scent.

7. The volatile liquid dispensing device of claim 1, wherein the volatile liquid dispenser is made of an absorbent material.

8. The volatile liquid dispensing device of claim 7, wherein the material has a thickness of at least about 0.20 inches.

9. A volatile liquid dispensing device comprising:
a body; and
a head, wherein the head comprises an engaging structure including an inner tab.

10. The volatile liquid dispensing device of claim 9, wherein the head has an incision cut into the head, the incision forming the inner tab.

11. The volatile liquid dispensing device of claim 10, wherein the incision extends to the edge of the head.

12. The volatile liquid dispensing device of claim 10, wherein the incision is in the shape of a spiral.

13. The volatile liquid dispensing device of claim 9, wherein the volatile liquid dispenser is made of an absorbent material.

14. The volatile liquid dispensing device of claim 13, wherein a volatile liquid is applied to the absorbent material.

15. The volatile liquid dispensing device of claim 14, wherein the volatile liquid is an animal scent.

16. The volatile liquid dispensing device of claim 15, wherein the material has a thickness of at least about 0.20 inches.

17. A method of dispensing a volatile liquid into a desired environment comprising:
applying the volatile liquid to a dispensing device that includes a head portion and a body portion, the head portion having a cut formed therein that permits a first portion of the head portion to be displaced relative to a second portion of the head portion;
displacing the first portion relative to the second portion to form a gap between the first and second portions;
interacting the dispensing device with a support structure present in the desired environment such that the support structure is present in the gap between the first and second portions;
engaging the support structure between the first and second portions such that the dispensing device is supported by the support structure; and
releasing the volatile liquid from the dispensing device into the desired environment.

18. The method of claim 17, wherein:
the first portion of the head portion includes an inner tab; and
engaging the support structure comprises projecting the inner tab against the support structure.

19. The method of claim 18, wherein interacting the dispensing device with the support structure comprises unwinding the head portion to form a hook-like structure and engaging the hook-like structure to the support structure.

20. The method of claim 19, wherein engaging the head portion with the support structure comprises winding the head portion around the support structure.

21. The method of claim 18, wherein interacting the dispensing device with the support structure comprises inserting a portion of the support structure through the cut in the head portion.

22. The method of claim 18, wherein engaging the support structure includes displacing the inner tab between the support structure and the head portion.

23. The method of claim 18, wherein the cut in the head portion is in the shape of a semi-circle to form the inner tab.

24. The method of claim 17, wherein applying the volatile liquid to the dispensing device comprises dipping the body of the dispensing device into a quantity of the volatile liquid.
